(19) United States
(12) Patent Application Publication
(10) Pub. No.: US 2026/0204432 A1
(43) Pub. Date: Jul. 16, 2026

(54) SYSTEM AND METHOD FOR PROVIDING HOSPITAL CUSTOMER RELATIONSHIP MANAGEMENT CONTENT

(71) Applicant: FLYINGDOCTOR INC., Goyang-si (KR)

(72) Inventor: Woo Jin LEE, Seoul (KR)

(21) Appl. No.: 19/138,640

(22) PCT Filed: Dec. 15, 2023
(86) PCT No.: PCT/KR2023/020766

§ 371 Date: Jun. 12, 2025

(30) Foreign Application Priority Data
Dec. 15, 2022 (KR) 10-2022-0175913
Dec. 15, 2023 (KR) 10-2023-0182655

(51) Int. Cl. G16H 80/00; G06Q 30/01; G16H 40/20

(52) U.S. Cl. CPC G16H 80/00; G06Q 30/01; G16H 40/20

(57) ABSTRACT

Provided are a method and device for transmitting CRM content from a medical institution client allocated to a hospital or a doctor. CRM content to be transmitted is selected from the medical institution client and is transmitted upon receiving an input of identification information about a receiver who is to receive the CRM content. Feed content created by a doctor is provided as at least a portion of the CRM content.

### TECHNICAL FIELD

The following description relates to a system and method for providing hospital customer relationship management content.

### BACKGROUND ART

Customer relationship management (CRM) refers to activities for conducting customer-tailored marketing by managing communication with customers. In recent years, even hospitals (hereinafter referred to as “medical institutions”) have adopted CRM practices by providing information to their customers, namely patients, through text messages or social networking services (SNS), and by collecting and processing patient inquiries, requests, and complaints for use in patient management. Such CRM activities in medical institutions are also used as a business strategy for increasing patient satisfaction through active communication, thereby ultimately improving visit and revisit rates.

Such CRM in medical institutions may include not only reservation information and hospital notices, but also medical or health-related information. In many cases, CRM involves hospital staff making phone calls, sending emails or text messages to patients, outsourcing to external services to send mass SMS/MMS messages (texting), or delivering messages via app-based push notifications or instant messaging services (such as KakaoTalk). Most of these are one-way, one-time messages.

(Patent Document 1) Korean Laid-Open Patent Publication No. 10-2019-0135692 (published on Dec. 9, 2019) discloses a hospital CRM system that extracts and processes information required by a patient using medical electronic medical record (EMR) data and transmits the information to the patient in the form of a message or provides it to a hospital terminal.

(Patent Document 2) Korean Registered Patent No. 10-1250462 (published on Apr. 8, 2013) discloses a hospital CRM system that enables a hospital to provide consultation services to existing patients based on electronic medical records and to identify marketing targets, i.e., potential medical service users, thereby actively attracting new patient customers.

(Patent Document 3) Korean Registered Patent No. 10-1875306 (published on Jul. 5, 2018) discloses a system for providing disease information using medical terminology clusters.

(Patent Document 4) Korean Registered Patent No. 10-0366816 (published on Jan. 9, 2003) discloses a remote medical device and method.

### DISCLOSURE OF THE INVENTION

### Technical Goals

A system is provided for customer relationship management (CRM), which serves as a communication channel to a hospital for patients by connecting the hospital with patients and enables the hospital to actively and proactively manage patients and potential patients.

The system also integrates the process of providing content, including medical information, to patients by physicians and/or hospital staff into CRM, to improve ease and convenience, and further provides a user-friendly experience to content creators to facilitate the content creation process.

The system allows medical images or illustrations to be conveniently used during content creation, thereby assisting in the creation and delivery of CRM content.

The process of delivering content to patients and customers is also improved for convenience, allowing patients to receive content of interest in association with their treatment, thereby supporting health management and treatment adherence.

Furthermore, the system contributes to enhancing trust between physicians (and hospitals) and patients, and to improving patient satisfaction with the hospital and the completeness of care throughout the entire care cycle.

### Technical Solutions

There is provided a computer-implemented method including providing a first interface configured to generate customer relationship management (CRM) content in association with a medical institution client assigned to a hospital or a physician; storing the generated CRM content in a storage unit; and transmitting the selected CRM content to a recipient based on a selection of the recipient and the CRM content to be transmitted.

According to one embodiment, the method may further include providing a second interface that is linked with the first interface and configured to provide a function of searching for and selecting an image to be inserted into the CRM content. In this case, results provided by the image search may be filtered based on at least one piece of tag information associated with the image.

According to one embodiment, the method may further include providing a third interface that is distinguished from the second interface and configured to receive a request to create a new image.

According to another embodiment, the method may further include extracting, by a processor, a tag for an image included in the CRM content while storing the generated CRM content in the storage unit.

By way of example and not limitation, the extracting may include obtaining, through a Bidirectional Encoder Representations from Transformers (BERT) model, at least one keyword for the image and extracting the obtained keyword as the tag. In this case, during training of the BERT model, a weight may be applied to text within a predetermined number of words following the image.

According to yet another embodiment, the method may further include providing identification information of a patient who has received treatment at the end of the treatment and receiving a selection as to whether to transmit the CRM content to the patient. If the CRM content includes a link for requesting remote treatment, and when the link is selected, a remote treatment request process is initiated.

### Effects of the Invention

According to embodiments, a licensed physician may leverage their medical expertise to provide expert medical columns to patients, including those who have visited the physician, potential patients interested in the physician's medical field, and other general subscribers.

During the process of writing texts (e.g., MobiDoc™ feeds), appropriate images may be easily inserted without infringing copyright, and a process is provided for evolving the image archive through communication with the MobiDoc™ team.

By publishing customer relationship management (CRM) content including such feeds and transmitting it to patients, the system connects physicians to patients as well as hospitals to patients. For patients, it serves as a channel for mutual communication with the physician or hospital, while for hospitals, it enables active and proactive management of patients and potential patients.

Furthermore, since MobiDoc™ feeds may only be authored by licensed physicians (thereby ensuring content reliability), patients receive accurate medical information. At the same time, physicians may reduce the need to repeatedly explain the same matters, thus preserving their valuable consultation time. From the patient's perspective, instead of passively receiving explanations during consultation, they are provided with created content conveying what their attending physician wishes to communicate. As a result, patients may read the content slowly and repeatedly, which leads to improved satisfaction with the physician, the hospital, and the overall treatment experience. This in turn may contribute positively to treatment outcomes.

Various other effects may also be provided by the embodiments and will be further described throughout the specification along with specific examples.

### BRIEF DESCRIPTION OF DRAWINGS

FIG. 1 is a diagram illustrating a connection among a customer relationship management (CRM) content providing device, a patient terminal, and a physician terminal.

FIG. 2 is a diagram illustrating a CRM content providing device according to one embodiment.

FIG. 3 is a diagram illustrating an example of CRM content according to embodiments, which is transmitted to a patient terminal and displayed via a patient client.

FIG. 4 is a diagram illustrating a physician client screen according to one embodiment.

FIG. 5 is a diagram illustrating a screen 500 displaying a list of created feeds.

FIGS. 6 to 9 are diagrams illustrating processes of creating feeds, searching for and selecting images, and requesting new images according to embodiments.

FIG. 10 is a diagram illustrating exemplary feed content.

FIGS. 11 and 12 are diagrams illustrating a process of extracting a tag from an image according to embodiments.

FIGS. 13 and 14 are diagrams illustrating a feed transmission process and features according to embodiments.

FIG. 15 is a diagram illustrating CRM content transmitted to a patient terminal according to one embodiment.

FIG. 16 is a diagram illustrating the provision of a remote medical service according to one embodiment.

### BEST MODE FOR CARRYING OUT THE INVENTION

Hereinafter, embodiments will be described in detail with reference to the accompanying drawings. When describing the embodiments with reference to the accompanying drawings, like reference numerals refer to like components and a repeated description related thereto will be omitted.

The terms used herein are selected from terms generally understood by those skilled in the related art but may have different meanings according to technical developments and/or changes, practices, and preferences of an engineer. Accordingly, the terms used herein should not be construed as limiting the technical spirit and should be construed as illustrative terms to describe example embodiments.

In addition, in a specific case, most appropriate terms are arbitrarily selected by the applicant. In this instance, the meanings of the arbitrarily used terms will be clearly explained in the corresponding description. Hence, the terms should be understood not by the simple names of the terms but by the meanings of the terms and the following overall description of this specification.

According to one embodiment, a customer relationship management (CRM) content providing device may be an electronic device. The electronic device may include any device that provides service connection and implementation, such as hardware in the form of a system-on-chip (SoC) and/or a general-purpose computing device implemented by application software.

For example, the electronic device may include at least one of a smartphone, a tablet personal computer (PC), a mobile phone, a video phone, an e-book reader, a desktop PC, a laptop PC, a netbook computer, a personal digital assistant (PDA), a portable multimedia player (PMP), an MP3 player, a mobile medical device, a camera, or a wearable device (e.g., a head-mounted device (HMD) such as electronic glasses, electronic clothing, an electronic bracelet, an electronic necklace, an appcessory, an electronic tattoo, or a smart watch).

According to additional embodiments, the electronic device may include at least one of various medical devices (e.g., magnetic resonance angiography (MRA), magnetic resonance imaging (MRI), computed tomography (CT), imaging devices, or ultrasonic diagnostic devices), navigation devices, global positioning system (GPS) receivers, event data recorders (EDRs), flight data recorders (FDRs), vehicle infotainment systems, marine electronic equipment (e.g., marine navigation devices or gyrocompasses), avionics, security devices, vehicle head units, industrial or home robots, automatic teller machines (ATMs) used in financial institutions, or point-of-sale (POS) systems used in hospitals.

In addition, the electronic device may include at least one of intelligent furniture for remote medical service connection, a part of a building or structure, an electronic board, an electronic signature receiving device, a projector, or various measuring instruments (e.g., water, electricity, gas, or electromagnetic wave measuring instruments). The electronic device according to various embodiments of the present disclosure may be any one or a combination of the above-described devices. The electronic device may also be a flexible device. As such, the form of the electronic device may vary, and it will be apparent to those skilled in the art that the form and primary purpose of the electronic device (excluding the remote medical service function) are not limited to the examples described above. Specific embodiments will be described below with reference to the accompanying drawings.

### CRM Service Provided by MobiDoc™ According to Embodiments

The MobiDoc™ service provides CRM. While satisfying the personalized needs of patients, MobiDoc™ takes a hospital-centered approach that distinguishes it from existing services. Just as patients seek physicians who may treat their conditions, physicians and hospitals also strive to attract and manage patients that best suit their medical services through various means.

For example, hospitals and physicians send text messages (such as treatment notifications, closure notices, hospital event/promotion notices, and appointment reminders) to encourage patients to revisit and use social networking services (SNS), such as KakaoTalk, to deliver messages. They operate websites introducing their hospitals and, more recently, have even invested time in managing blogs and YouTube channels. However, these activities incur significant costs, such as for message transmission and promotional agency services, and there is no service optimized specifically for physicians and hospitals.

MobiDoc™ provides advantages in these aspects. When using the CRM functionality provided by MobiDoc™, client interfaces on the hospital side (i.e., a client used by physicians through login and clients used by hospital staff through login) may collaborate with one another and complement each other's roles. Accordingly, when a physician wishes to provide special care to a patient while scheduling a follow-up appointment immediately after a consultation, CRM content may be created or edited and applied to that patient. Hospital staff may also configure CRM content in a time-efficient manner.

Accordingly, delays in medical practice by healthcare professionals may be reduced, and hospital operations may proceed smoothly even in hospitals with a shortage of staff relative to workload or during temporary surges in patient volume. Furthermore, compared to using mass-texting agent services that transmit CRM content via SMS or MMS, the system enables more sophisticated customization tailored to individual customers and hospitals, allows for more proactive responses to changing circumstances, and offers improved cost competitiveness.

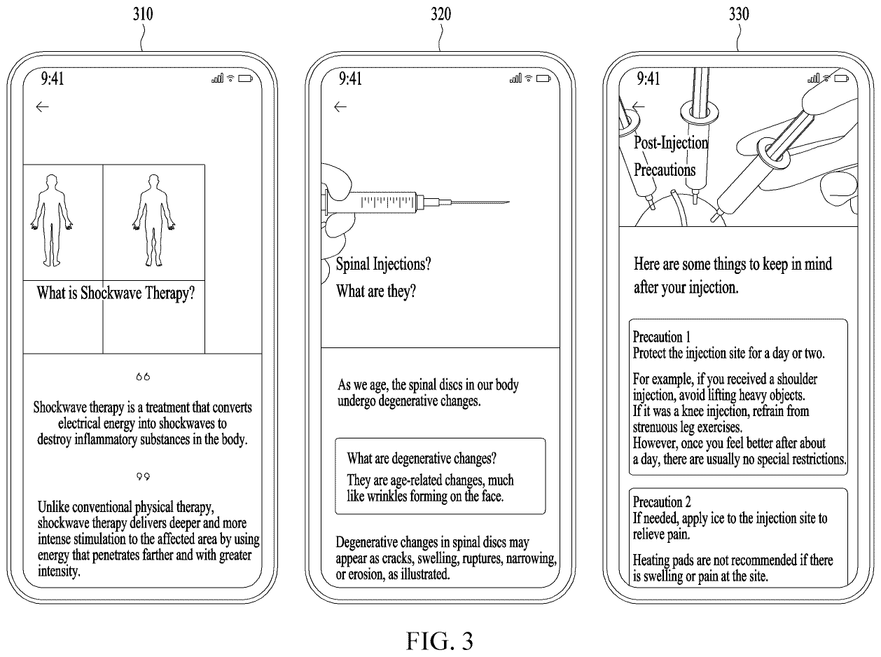

In addition, the CRM provided by MobiDoc™ may include various types of content delivered to patients, such as appointment creation notifications, reminders before the appointment date, same-day appointment reminders, examination instructions, directions to the hospital, physicians'instructions, digital prescriptions after consultation, digital therapeutics, records/guides for monitoring treatment progress, post-treatment or post-procedure care instructions, MobiDoc™ feeds authored by physicians as expert medical columns, and notifications and links for remote treatment requests. Types of CRM content, as well as the processes for content creation, management, and transmission, will be described in more detail with reference to FIG. 3 and subsequent figures.

### System and Service Architecture

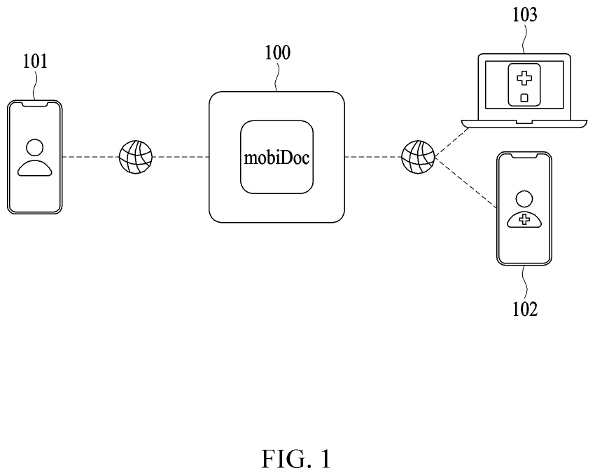

FIG. 1 is a diagram illustrating a connection among a CRM content providing device 100, a patient terminal 101, and physician terminals 102 and 103 belonging to a medical institution.

The device 100 in the illustrated diagram may be implemented by a server-grade computer, a workstation, a desktop computer, a laptop computer, or a mobile electronic device, and may also be implemented by a combination of two or more of these devices.

The device 100 may be connected to the patient terminal 101 via a network, and also to the physician terminal 102 and the hospital terminal 103. Each of the terminals 101 to 103 may correspond to a desktop computer, a laptop computer, or a mobile electronic device, such as a smartphone or a tablet PC. Each of the terminals 101 to 103 includes software means, referred to as a “client” for convenience of description.

The patient terminal 101 executes a patient client. The physician terminal 102 and the hospital terminal 103 execute a physician client and a hospital client, respectively. The physician client serves as a counterpart to the patient client and is installed in a hospital. A cloud-based service in the form of Software as a Service (Saas) may also be provided. In this case, the physician client refers to a cloud service that is accessed through an account of a physician and/or hospital staff. The hospital client refers to a means for performing medical support tasks, such as processing consultation requests or hospital payments, by administrative staff or nurses, rather than by medical professionals (physicians) who provide medical treatment.

A physician client used by physicians may be provided separately from a hospital client 103 used by hospital staff other than physicians. However, according to one embodiment, the physician client and the hospital client may be integrated into a single client. In this case, when the sign-in account belongs to a licensed physician, all functions may be activated, whereas when the sign-in account belongs to a staff member who is not a physician, certain functions may be deactivated. The deactivated functions may include, for example, functions such as “start remote treatment,” which are to be performed only by physicians. Since the appropriate timing for initiating the next treatment for a patient may be most accurately and appropriately determined by the physician, the “start remote treatment” function, which initiates a video connection with a patient client waiting in a consultation room, may be implemented as a function operable only by physicians.

In addition, according to embodiments, certain content creation processes, such as creating MobiDoc™ feeds, which are intended to be authored exclusively by physicians, may be permitted only when accessed through a physician account. However, this is merely illustrative. Hereinafter, it may be understood that the term “medical institution client,” as used throughout this specification, collectively refers to both the physician client and the hospital client installed respectively on the physician terminal 102 and the hospital terminal 103.

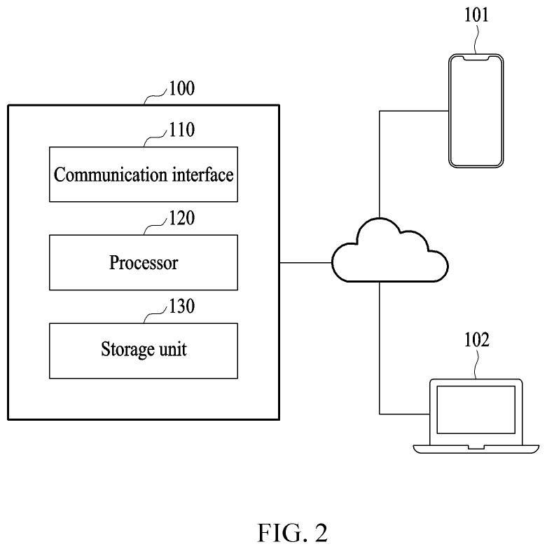

Clients installed on the terminals 101 to 103 may be original application software programs, web-based services accessed through a browser page, or hybrid applications incorporating both app and web functionalities. Furthermore, they may be implemented as cloud-based services provided in the form of SaaS. These implementations may be readily achieved by those of ordinary skill in the art, and thus a detailed description thereof is omitted. Although the embodiments described herein may refer to a specific implementation example, such reference should not be construed as excluding other implementations. The implementation and operation of the device 100 are described with reference to FIG. 2.

### System Architecture and CRM Content Transmission

FIG. 2 is a diagram illustrating a CRM content providing device 100 according to one embodiment. The device may be an electronic device including at least one processor 120, a communication interface 110, and a storage unit 130. The device may be communicatively connected to a patient terminal 101 and a medical institution (hospital) terminal 102 and may provide a service of transmitting CRM content from a client executed on the medical institution-side terminal to the patient-side terminal.

According to one embodiment, a method for providing CRM content by the device 100 includes providing a first interface configured to generate CRM content in association with a medical institution client assigned to a hospital or a physician; storing the generated CRM content in a storage unit; and transmitting the CRM content to a recipient based on a selection of the recipient and the CRM content to be transmitted.

According to one embodiment, the method may further include providing a second interface that is linked with the first interface and configured to provide a function of searching for and selecting an image to be inserted into the CRM content. In this case, results provided by the image search may be filtered based on at least one piece of tag information associated with the image.

According to one embodiment, the method may further include providing a third interface that is distinguished from the second interface and configured to receive a request to create a new image.

According to another embodiment, the method may further include extracting, by a processor, a tag for an image included in the CRM content while storing the generated CRM content in the storage unit.

### CRM Content Transmission Path

A CRM content transmission request includes identification information of a recipient who is a target of the CRM content transmission and may trigger a process for verifying whether the MobiDoc™ app has been installed. By way of example and not limitation, this verification may be performed by checking demographic information, a customer number, or a phone number of the patient in a database (DB) of the storage unit 130. If the app is determined to be installed, the CRM content may be transmitted via a first means. The first means may be, for example, push message transmission through the MobiDoc™ app. However, if it is determined that the app has not been installed on the patient terminal, the CRM content may be transmitted via a second means. The second means may include, for example, a text message or an instant messaging service, such as KakaoTalk.

### MobiDoc™ Feed Content

FIG. 3 is a diagram illustrating an example of CRM content according to embodiments, which is transmitted to a patient terminal and displayed via a patient client.

For example, the transmitted content may be explanatory information about the treatment received on the day, following an in-person visit to the hospital and/or an online video consultation.

A patient who visits the hospital and receives extracorporeal shock wave therapy may wonder what the therapy does and why it is beneficial, even though it was prescribed by a physician and administered in the physical therapy room. In typical clinical settings, medical staff such as physicians or physical therapists provide explanations, but the quality and clarity of these explanations may vary depending on the individual. Moreover, due to time constraints, the explanations may not always be sufficiently thorough. Patients may also forget what was explained during the consultation or while receiving physical therapy (or sometimes a procedure), especially when they are distracted or overwhelmed. Some patients, depending on their personality, may feel hesitant to ask questions even when they have concerns. MobiDoc™ addresses these issues and benefits both healthcare providers and patients. It allows sufficient information to be delivered to patients while saving time for medical staff. By simply checking the feed sent by the hospital after the consultation, patients may gain a clear understanding of the details of the treatment they received that day.

A screen 310 illustrates an example in which a physician who frequently administers extracorporeal shock wave therapy pre-writes a feed addressing questions that patients are likely to have and sends the feed to a patient who received the therapy that day. A screen 320 illustrates an example in which a feed explaining a spinal injection and the related condition is pre-created and sent to a patient who received the spinal injection that day. A screen 330 displays precautions to be taken after injection treatment.

For reference, the MobiDoc™ Feed is a content delivery service provided by licensed medical professionals who are registered users. It is accessible through a physician client or the separate MobiDoc™ web platform and is intended to deliver content to patient users who follow the physician or the affiliated hospital. The MobiDoc™ Feed, by way of example and without limitation, may include content such as text, images, or videos relating to disease information, treatment trends, medical knowledge, or health management. A physician user may prepare such feeds in advance, based on their specialties such as diseases, procedures, or treatment methods, and selectively send them to patients as needed during consultation.

Meanwhile, the feed may also include a direct link for making a MobiDoc™ appointment with the physician who authored the feed or with a hospital or physician recommended by that physician for the relevant condition. If a patient has undergone a procedure or treatment-such as physical therapy, injection therapy, or laser therapy-the MobiDoc™ Feed may be used as a means of transmitting CRM information tailored to the patient, delivering additional details about the condition, future treatment plans, explanations of the treatment or procedure performed on the day, precautions, and/or detailed information regarding prescribed medications.

### Other Examples of CRM Content

Although FIG. 3 illustrates an example of the MobiDoc™ Feed, CRM content may include various examples.

For example, in the case of outpatients, guidance on the schedule for the next visit may be provided through the CRM content after the consultation is completed. For patients with an initial appointment, a notification regarding the date and time of the first visit may be provided. To help busy professionals, elderly individuals with memory impairments, or parents occupied with childcare avoid missing a scheduled treatment or vaccination, alerts may be sent regarding upcoming appointments or vaccination dates. In addition to merely notifying patients of scheduled appointments, the CRM system may also enable patients to check whether a change in the reservation is necessary and submit a request for rescheduling to the hospital.

Guide notification CRM content may be transmitted on the day of the appointment (D-0), or a few days in advance, such as D-7, D-3, or D-1, depending on the schedule. In addition to simply notifying patients that their appointment is one or three days away, the CRM content may also provide pre-examination instructions, such as medications that need to be discontinued or the required fasting period for patients who are scheduled to undergo checkups or tests.

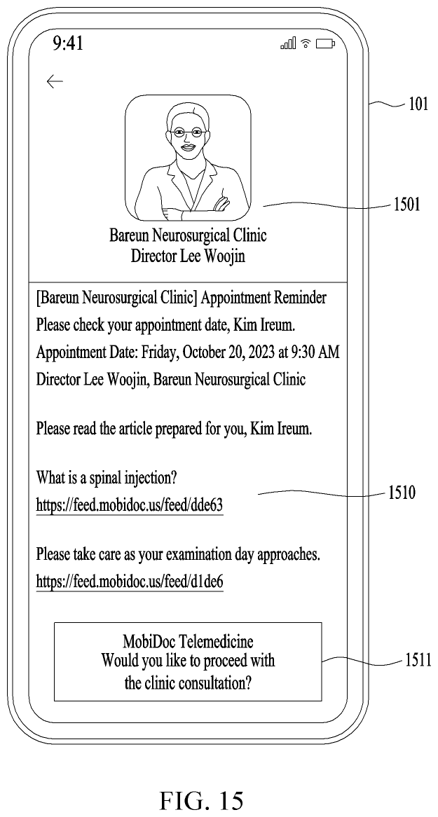

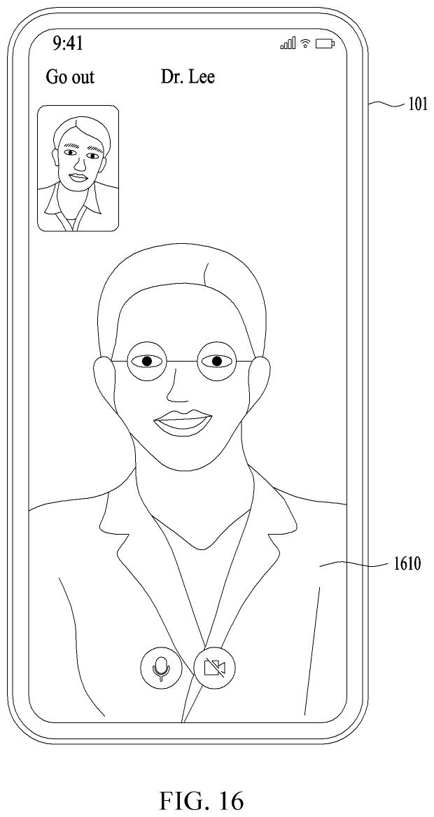

In medically appropriate cases-such as follow-up consultations where the physician only needs to assess the patient's progress and determine whether to adjust prescriptions, or cases where the main purpose of the visit is to explain the results of pre-conducted tests and issue prescriptions-the patient may be guided to receive a remote video consultation (MobiDoc™ Clinic), as illustrated in FIGS. 15 to 16. In such cases, the hospital side may initiate a CRM message informing the patient that the scheduled consultation for the day is suitable for a video consultation and inquiring whether the patient would like to switch to a remote format. Conversely, it is also possible for the patient, who originally planned to visit the hospital in person, to send a CRM message to the hospital requesting to switch the appointment to a remote video consultation (MobiDoc™ Clinic).

In another embodiment, a survey on treatment satisfaction may also be provided as CRM content. In addition to a survey on overall satisfaction, the CRM content may include objective and/or subjective questions regarding the physician seen during the visit, the physical therapist, hospital staff, treatment costs, and consultation time. Such treatment satisfaction surveys delivered via CRM may serve as valuable reference data for hospital administrators. Patients may also feel more positive about the hospital, recognizing that it is actively checking their level of satisfaction through such CRM interactions. Compared to the conventional experience of receiving promotional CRM messages from hospitals, the CRM service according to embodiments provides more immediate and improved customer experience.

According to yet another embodiment, a D+Day CRM may be provided not on the day of the treatment but during a post-treatment period, to follow up with the patient and deliver relevant information. For example, in dermatology, post-treatment care may require scheduled guidance: on the day after the procedure, patients may need to be informed of precautions and actions to take; on the third day, additional information may need to be provided; and on the seventh day, the system may prompt a checkup, instructing patients to contact the hospital if any abnormalities are observed. Traditionally, most dermatology clinics have provided such guidance in printed form, which has often caused inconvenience. Such needs are not limited to dermatological procedures but also arise in various medical interventions, including plastic surgery, other surgical operations, pain interventions, cardiac catheterization procedures, and other medical treatments for specific symptoms.

According to yet another embodiment, CRM content may also include modules such as digital prescriptions, digital therapeutics, and condition-specific surveys (e.g., the Visual Analogue Scale (VAS)) provided through the MobiDoc™ app. Unlike conventional healthcare apps that require users to separately install and manually operate them to record health data such as weight, blood pressure, or blood glucose, the MobiDoc™ CRM enables healthcare providers to directly prescribe customized modules within the MobiDoc™ platform. Specifically, the physician who provided the consultation may, through the MobiDoc™ physician client 102, assign and activate specific modules in the patient's MobiDoc™ account, specifying the duration, recording interval, and survey parameters. These modules are then executed on the patient terminal 101 precisely as prescribed, automatically recording designated metrics or presenting assigned surveys as a seamless extension of the consultation or treatment.

Such modularized CRM contributes to enhancing emotional stability for patients and improving the completeness of care. In particular, the fact that a hospital follows up with patients after they return home from outpatient visits-caring for outpatients as if they were inpatients-and, when necessary, proactively checks on their condition and provides appropriate recommendations, represents a new type of medical service enabled by the MobiDoc™ CRM according to the embodiments. This kind of customer experience (CX), which resembles being medically supported by a family member or close acquaintance, not only aids in patient recovery and helps prevent risk situations but also increases patient satisfaction and loyalty toward the hospital. From the hospital's perspective, such CRM also contributes to improving patient satisfaction and increasing revisit rates. Furthermore, in general hospitals or tertiary care centers where there is a chronic shortage of hospital beds and mild cases must be discharged quickly, the MobiDoc™ CRM enables continued patient management even after discharge. Accordingly, it supports outpatient monitoring and helps manage a patient's health while reducing hospitalization rates.

### Configuration of Physician Client and Creation of Feed Content

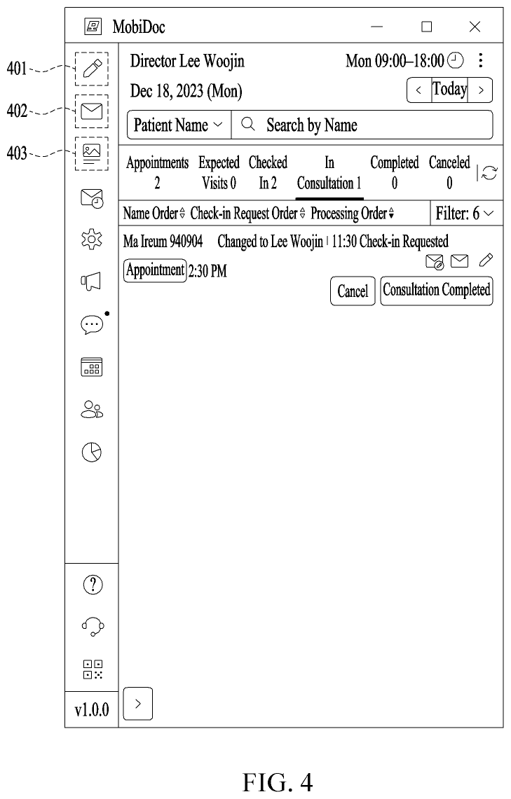

FIG. 4 is a diagram illustrating a physician client screen according to one embodiment. The MobiDoc™ physician client may be used by a physician during remote consultations or in-person visits to provide CRM content to patients while enhancing the completeness of care.

When the physician has access to the physician client 100, such as during a consultation or when there is spare time between consultations, the process of creating a feed may be initiated by pressing a “Write” button 401 on the physician client 100. In this case, the physician may select the writing function either within the app or through the MobiDoc™ web interface (not shown) to begin creating a feed. Of course, it is also possible to directly access a separate MobiDoc™ webpage, outside of the physician client 100, and select the “Write” button 401 to initiate feed creation.

When the physician client and the hospital client (used by staff such as nurses or administrative personnel) are integrated into a unified interface, the feed creation (writing) function is enabled only when logged in with an authenticated physician ID. For other user IDs, the feed creation function may not be displayed, and only the CRM sending function may be activated.

Previously published (sent) feeds 402 by the physician may be viewed through the MobiDoc™ webpage or the MobiDoc™ physician client app. In addition, an image required when creating a feed may be requested from MobiDoc™ using an “Image Request” button 403.

According to these embodiments, when there is a gap between consultations, the physician may create and store writings (feeds) from time to time, such as spontaneous insights, messages they wish to convey to patients during treatment, or information that typically needs to be repeatedly explained or clarified to patients. These feeds may then be published (sent) to selected recipients, thereby helping to maintain and strengthen the physician-patient relationship and enhancing the hospital's completeness of care for the patient.

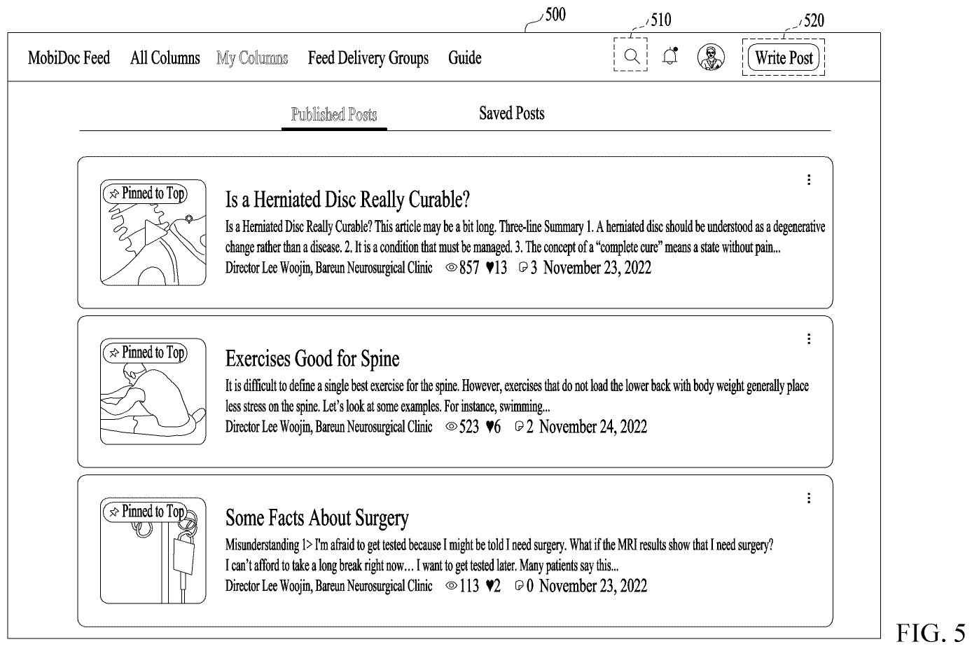

FIG. 5 is a diagram illustrating a screen 500 displaying a list of created feeds.

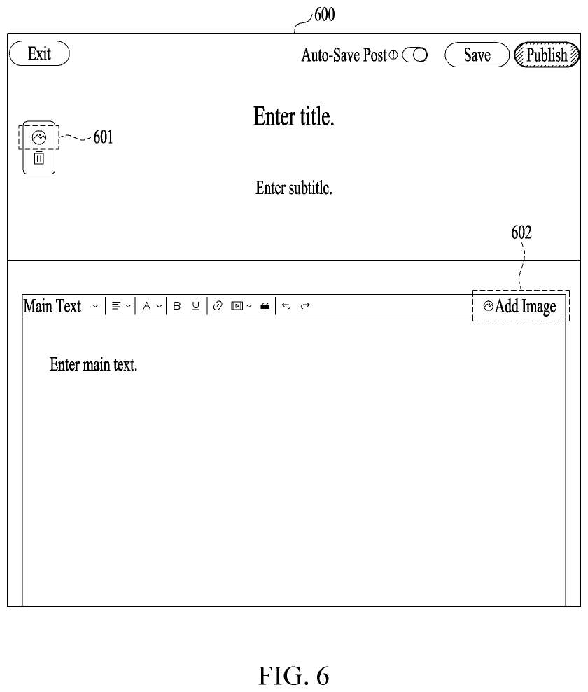

A “Search” button 510 is provided for retrieving previously created and published feeds intended to be sent to patients, and a “Write” button 520 is provided for composing a new feed. When the “Write” button 520 is pressed, the feed creation process, described with reference to FIG. 6 and the following figures, is initiated.

FIG. 6 illustrates a screen 600, which is used to initiate feed creation. On this screen, the physician may enter the title and subtitle of the feed and may insert a cover image using a button 601. The physician may also freely enter text into the editor window. The interface is similar to that used when creating posts on social networking services (SNS) or writing new blog entries. However, the embodiments provide distinctive advantages, such as improved convenience when selecting the cover image insertion button 601 or the body image insertion button 602 and searching for and inserting images, as well as relieving the writer from concerns related to image copyright.

According to embodiments, the distinctive service provided by MobiDoc™ in this case is the provision of an archive of pre-prepared images and illustrations that may be useful for creating medical feeds. This archive is stored and managed in the storage unit 130 of the device 100.

Feed postings, which are content containing medical information on the MobiDoc™ client, are created by physicians who are medical professionals. However, individual physician users may not always have sufficient access to appropriate images, and general illustrations or photographs may be unusable due to copyright restrictions. Furthermore, in the case of medical images, they may contain patients'personal information and therefore may not be freely used in feed creation.

Accordingly, according to embodiments, MobiDoc™ builds an archive of medical images that have either been produced in-house or obtained through licensed copyright, and that have been processed to remove any personal information. This content is provided to ensure that individual physician users of MobiDoc™ may use such images and videos without any concerns.

Physicians may select and insert images for the cover or body text by choosing the image insertion button 601 or 602 at the desired time and location during the writing process.

### Feed Image Search and Selection Interface

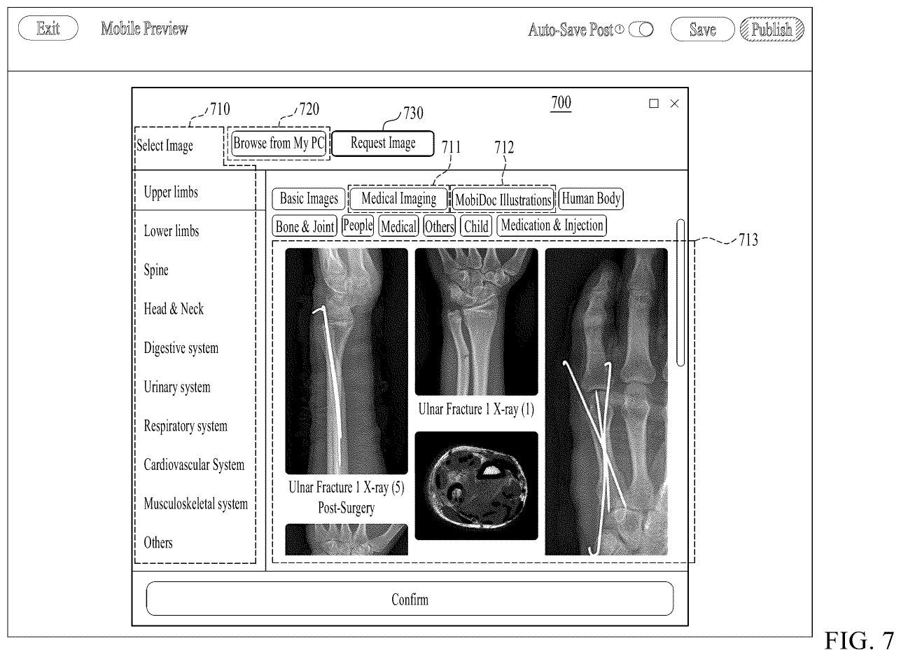

FIG. 7 illustrates a process of searching for, selecting, and inserting images during feed creation, provided according to one embodiment.

While creating a feed, a physician selects the image insertion button 602, upon which an image selection window 700 pops up, according to one embodiment.

One of the distinctive functions provided by the embodiments is the provision of a group selection interface 710, which displays images on the left side grouped according to certain criteria, for example, by body part, such that the physician may intuitively and easily select images to be inserted.

Selectable groups may include a wide range of categories, such as body parts (positions) including the whole body, upper limbs, lower limbs, spine, head and neck, digestive system, urinary system, respiratory system, cardiovascular system, and musculoskeletal system; body functions (roles); types of diseases; and behavior classifications related to human activities (e.g., exercise, sleep, driving, teeth brushing, etc.).

In addition, according to one embodiment, depending on the group selected by the physician, a subset of the stored illustrations, videos, or images may be selectively displayed at a viewing area 713. In this regard, the group selection interface 710 may also function as a filtering interface.

On the group selection interface 710, a primary group may first be selected, and additional tags for secondary filtering may be displayed on the right side. For example, a tag 711 may correspond to actual medical images such as basic images, X-ray images, or CT/MRI images, while a tag 712 may represent MobiDoc™ illustrations, which are custom-drawn and stored by MobiDoc™ based on specific body parts or diseases. Accordingly, the physician creating the feed may quickly and easily search for and select the appropriate image to use in the post. Since the images are either created in-house by MobiDoc™ or legally acquired with proper copyright ownership, the physician may intuitively select and use the desired illustrations without concerns about copyright issues. Of course, a button 720 may also be provided to allow the physician to retrieve images stored on their personal computer, if necessary. In addition, if the physician determines that no appropriate image is available either on their PC or in the MobiDoc™ archive, they may submit an image request to MobiDoc™ by using an image request button 730, asking for a new illustration to be created.

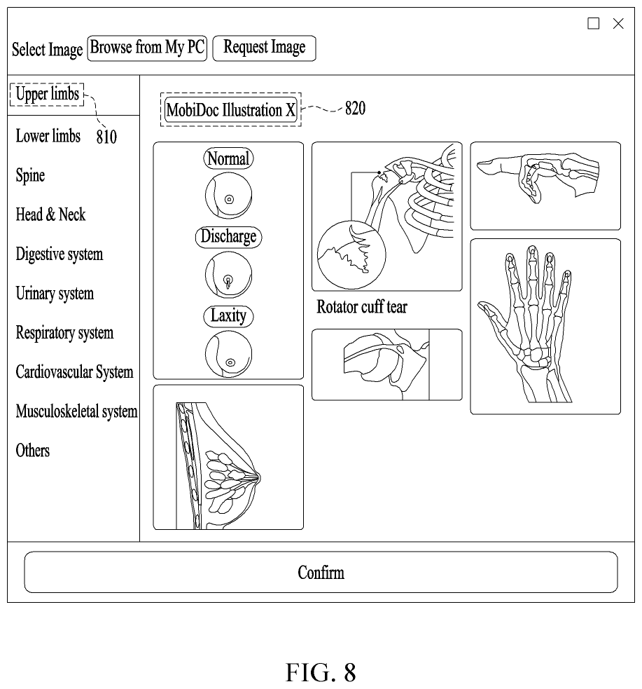

FIG. 8 illustrates a process of presenting and selecting images according to one embodiment, as described above. When the user selects an “upper limb” group 810 on the left side, an overview screen related to the upper limb is presented. When the MobiDoc™ illustration option 820 is selected on the right side, only illustrations created and provided by MobiDoc™, rather than medical images or general pictures, are displayed for selection.

In this case, the physician composing the feed may select an illustration corresponding to the “upper limb” area, appropriate for the purpose and content of the feed, without concerns about copyright infringement. Frequently used images may also be marked as favorites, allowing the physician to easily access and reuse them later.

### Feed Image Request

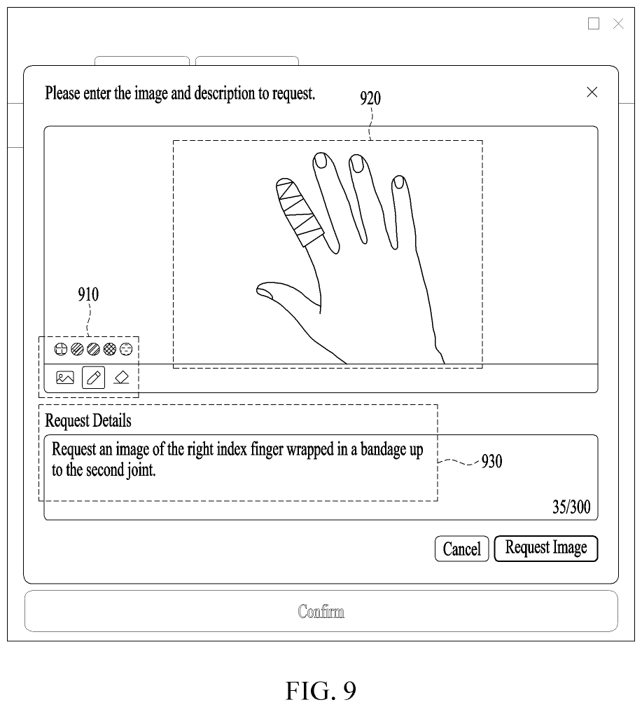

Meanwhile, in some embodiments, as previously described, when a physician determines that an appropriate image is not available either on their PC or in the MobiDoc™ archive, they may request the creation and provision of the image by pressing the image request button 730. FIG. 9 illustrates a process, according to one embodiment, in which a physician creating feed content submits a request to MobiDoc™ by describing the required image.

When a physician describes the desired image and submits an image request to MobiDoc™'s illustration artists, the artists may create the requested image, store it in the MobiDoc™ archive, and provide it to the requesting physician. The request may be made using an interface 910, which allows the physician to either upload a reference image or draw a rough sketch. If necessary, the physician may directly draw in the drawing field 920, which functions similarly to a paint application that supports image pasting and freehand drawing. To provide further guidance, the physician may enter specific requirements in an input field 930, which the artists may refer to during the illustration process.

### Manual and Automated Tagging of Feed Images

Meanwhile, according to some embodiments, the filtering tags described above may be generated and assigned to each image at the time of creation via an image request. This is intended to facilitate convenient and accurate retrieval of images from the image archive in the future, similar to how hashtags are used in posts or images to enable later search functionality.

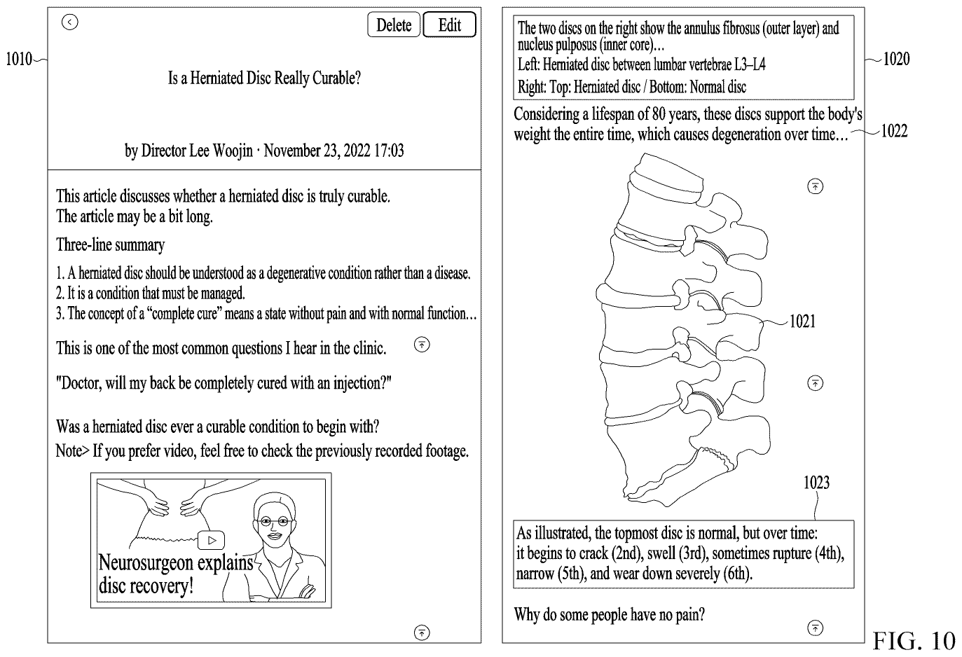

FIGS. 10 to 12 are diagrams illustrating a process of manually and/or automatically extracting image tags from a feed being created or already created, to be used for future image searches, according to embodiments.

FIG. 10 illustrates a feed that has been created and published. A preceding area 1010 and a following area 1020 represent parts of a single feed. In area 1010, the title “Is a herniated lumbar disc truly curable?”, along with a summary and main body text, are included, based on which a worker may manually extract relevant tags. For example, such tags may include “disc herniation” and “lumbar”.

From the right area 1020, tags for the image 1021 may be extracted from the main text preceding 1022 or following 1023 the image 1021. During the initial creation of the image by a MobiDoc™ artist at the request of a physician, provisional tags may also be assigned. These provisional tags may then be reviewed and confirmed by an expert, such as the requesting physician, and subsequently stored as finalized tags.

Meanwhile, another distinctive feature of MobiDoc™ according to embodiments is its image tag extraction functionality. Assigning filtering properties such as hashtags to “MobiDoc™ illustrations” created by MobiDoc™ artists at the request of physicians is, by default, a manual process. As such, it requires considerable resources. Moreover, because only medical professionals with appropriate expertise may reliably finalize hashtag assignments, manual-only processing may result in limitations in classification consistency, accuracy, and processing speed. Since inaccurate tagging may hinder later image retrieval and use, the tag assignment process is critical. Therefore, according to embodiments, the extraction and assignment of tags are at least partially supported by artificial intelligence (AI) technology.

According to one embodiment, the implemented device 100 may automatically extract hashtags by using AI-based image recognition of the MobiDoc™ illustration itself (e.g., the image 1021) and/or semantic keyword extraction from surrounding text (e.g., the area 1022 or 1023). This reduces the resources required for hashtag assignment at the time of image registration and enables easier retrieval of relevant images during content creation.

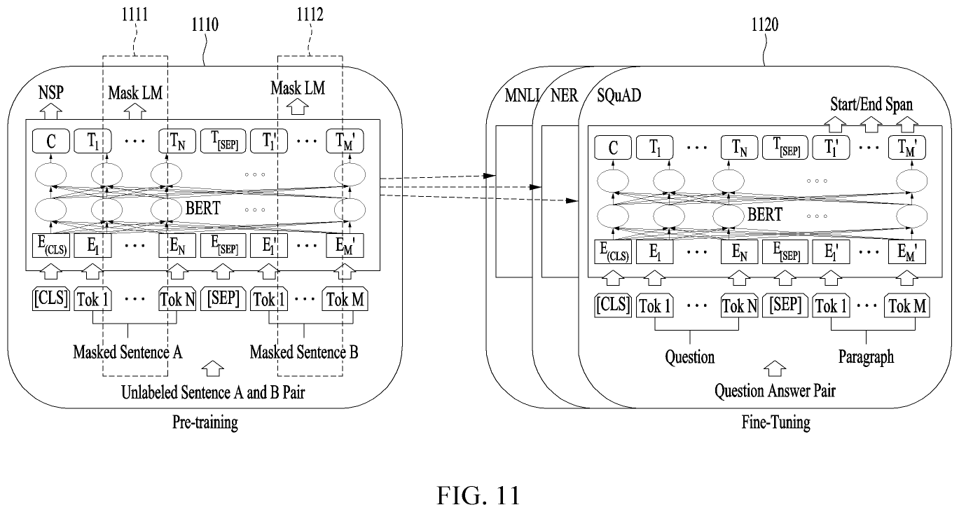

Various models may be used to extract keywords that serve as tags from images and/or feed content; however, according to one embodiment, the processor 120 of the device 100 uses Bidirectional Encoder Representations from Transformers (BERT). BERT is a transformer-based machine learning technique developed by Google in 2018 for natural language processing. Its key feature is the ability to understand context by learning sentence structure bidirectionally. This characteristic has been found to be well-suited for use with medical images and medical illustrations according to embodiments. FIG. 11 illustrates this bidirectional learning process.

A core mechanism enabling BERT's bidirectional learning is a masked language model (MLM), which is applied during a pre-training phase 1110. MLM involves predicting keywords within a given sequence of text. To train the model for bidirectional context understanding, a certain percentage of input tokens are randomly masked, and the model is trained to predict the original tokens that were masked.

The mechanisms and processes 1111 and 1112 of MLM are as follows. 15% of the tokens in the entire sequence are randomly selected for masking. Among these selected tokens, 80% are replaced with [MASK] tokens, 10% are replaced with random words, and the remaining 10% are left unchanged. This process is incorporated into a fine-tuning phase 1120, during which training is performed.

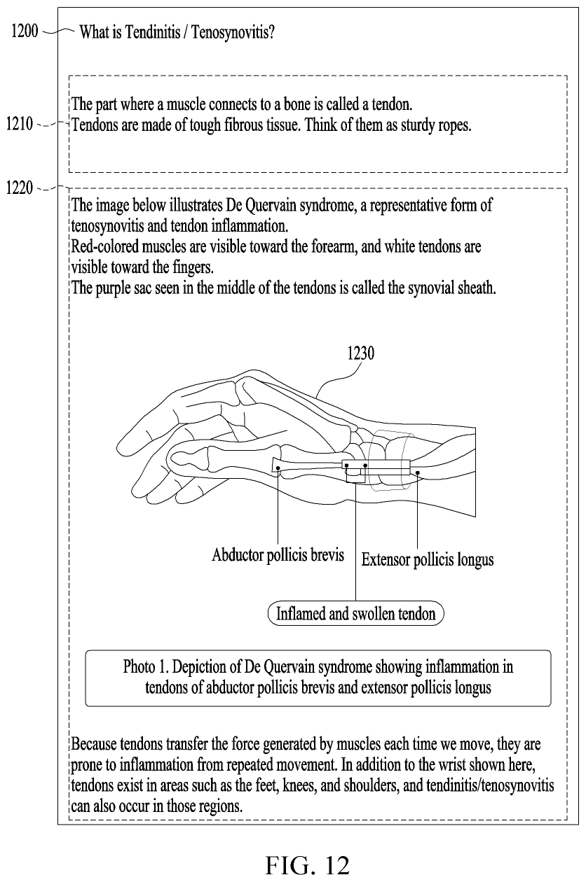

One detailed example of testing on a medical image is described with reference to FIG. 12. Texts included in feed posts 1210 and 1220 related to tenosynovitis were trained as follows:
Original: The part where a muscle attaches to the end of a bone is called a tendon;
(80%) The part where a muscle attaches to the end of a bone is called [MASK];
(10%) The part where a muscle attaches to the end of a bone is called skin; and
(10%) The part where a muscle attaches to the end of a bone is called a tendon.

When applying this model, keywords required from the overall context were successfully extracted; however, it was confirmed that they were not appropriate as hashtags for an actual image 1230 included in that area. In particular, when a feed contains a plurality of images, the appropriateness was observed to decrease further. Accordingly, according to embodiments, a higher weight is assigned to sentences near the image-namely, the sentence immediately before or after the image, especially the one immediately after. Keywords extracted from the entire text are assigned a lower weight and retrained accordingly. In this exemplary experiment, the accuracy of hashtag extraction improved significantly by applying this method. In the example of FIG. 12, the words ‘tendon’ and ‘de Quervain's disease’ were extracted for the image 1230. This level of automatic extraction may serve as a sufficiently reliable draft for future review by medical professionals.

### Publication (transmission) and Management of Feeds

Feeds published after creation are basically managed as part of the corresponding physician member's feed library. As originally intended, the feeds may be transmitted to customers who follow the physician (existing patients, general MobiDoc™ users, or specific individuals selected by the physician to receive the feed).

By using the MobiDoc™ feed provided by the MobiDoc™ platform, physicians may obtain various benefits. As described above, when the consultation time assignable to a patient is limited due to a large number of waiting patients, the physician may provide a pre-created feed to the patient to deliver additional information regarding a disease or treatment, thereby securing sufficient patient education while reducing consultation time. From the perspective of a patient who lacks medical knowledge, a feed that may be reviewed slowly and repeatedly at a later time may be more helpful than a brief oral explanation delivered rapidly during a short consultation. Accordingly, the use of such feeds may enhance trust and loyalty between the physician and the patient.

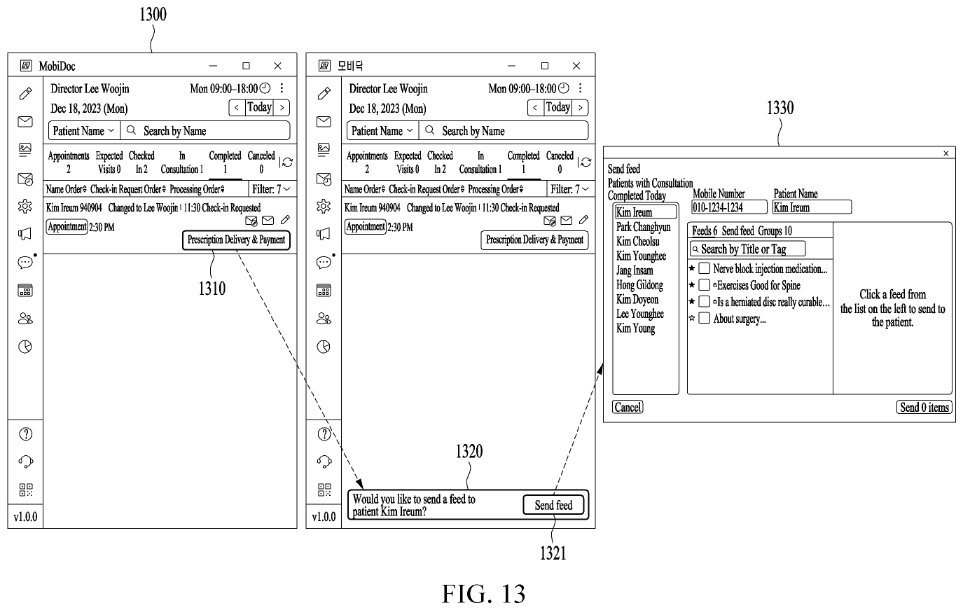

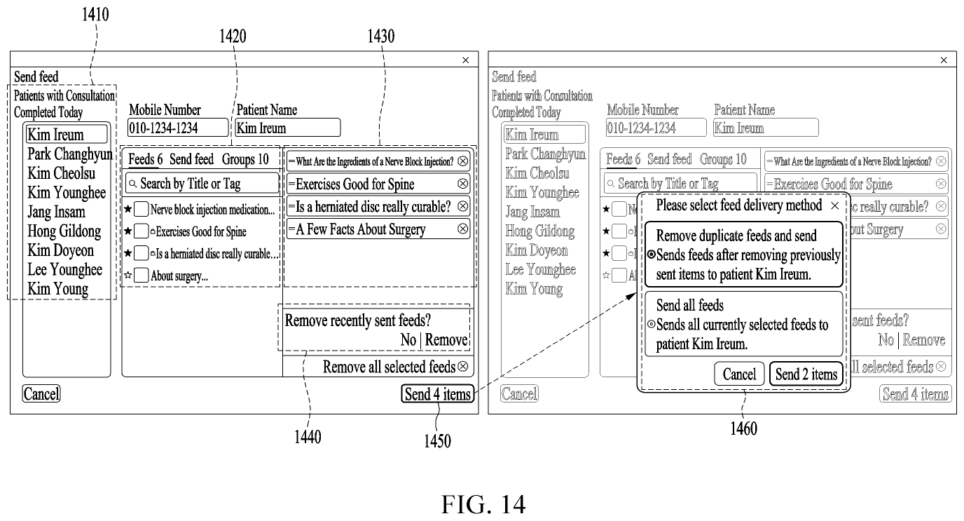

According to one embodiment, as illustrated in FIG. 13, when treatment is completed on a physician client 1300 and an area 1310 for prescription delivery and payment is selected, a pop-up window 1320 may be displayed to inquire whether to send a feed to the patient who has just been treated. If an area 1321 for transmitting the feed is selected, a feed selection and transmission pop-up window 1330 may be provided to assist in sending the feed.

In an interface for selecting a patient to receive a feed, “patients who have just been treated” may be listed and presented in chronological order (most recent first), allowing the physician to promptly identify the most recently treated patient and transmit an appropriate feed. In addition, the interface may include a separate option for selecting the “patient currently undergoing treatment”, thereby enabling the physician to send a suitable feed either before or immediately after the treatment is completed.

For reference, based on various patient voices of customer (VOC), it is often found that patients fail to recall or misunderstand the physician's explanations or precautions provided during consultation regarding their conditions. The MobiDoc™ system assists physicians in continuously providing care to patients while efficiently conserving their consultation time, thereby enhancing patient satisfaction and building trust in the physician and the medical institution. In this respect, the MobiDoc™ feed functions as a key component of hospital CRM.

When the “Send Feed” function is used on the physician client, either the selection of the recipient or the selection of the feed to be sent may be performed first.

When selecting a recipient (customer or patient), the physician may enter a portion of a remembered mobile phone number to filter and select patients through search/autocomplete (recommendation). Alternatively, the physician may begin typing the patient's name, and relevant search results may be presented dynamically to enable selection without fully entering the name. In the case of duplicate names, additional patient information (e.g., residence, age/gender, diagnosis, visit date, appointment date, or notable characteristics) may be displayed to assist the physician in making the correct selection. Timely recommendations may also be made through separate interfaces, such as those for recently seen patients, patients who have just left, patients seen today, patients with appointments today, or patients scheduled for a visit tomorrow.

Furthermore, it is possible to group patients and select them as a group, such as “patients with cervical disc disease at our hospital,” “patients who have revisited our hospital more than twice,” “patients subscribed to my column,” or “patients residing in Donae-dong, Deokyang-gu, Goyang-si.” Just as a plurality of feeds may be selected, a plurality of patients may also be selected.

Sending the same feed again to a patient who has already received it may be considered spam-like. Accordingly, if a patient is selected and a feed is selected, and the selected feed has already been sent to the selected patient, a warning message such as “This feed has already been sent to this patient. Do you still want to send it?” may be provided to prevent accidental transmission. Options may also be provided to exclude previously sent feeds from a group of selected feeds or to exclude specific patients from the list of recipients for feeds they have already received.

As a specific example, according to one embodiment, when a patient to receive a feed is selected in an area 1410 and a feed to be sent is selected from a list 1420, a list 1430 of previously sent feeds to the selected patient may also be displayed. This allows the physician to check whether the selected feed has already been delivered to the patient. In some cases, a pop-up 1440 may be provided to confirm whether to resend a feed that was recently delivered. After this process, even when the physician selects a feed sending area 1450, if any duplicate feeds exist, the device 100 may prompt in an area 1460 whether to exclude such duplicate feeds before transmission. Subsequently, actual transmission of the feeds may be performed.

### Content That may be Included in Feed and its Applications

FIG. 15 illustrates an example of a feed screen displayed on the patient terminal 101 after transmission.

In addition to the foregoing images, the feed may also display YouTube videos (see the area 1010 of FIG. 10), the physician's personal blog, or hospital website information (in partnership with MobiDoc™), thereby enabling the physician to continuously attract returning patients.

Furthermore, within the published feed, communication may occur between the physician and patient or among patients through comments or direct messages (DMs) directed to the authoring physician. Patients may also request a consultation with the physician via the MobiDoc™ clinic or follow the physician, thereby allowing the physician and hospital to build relationships with patients.

According to embodiments, various forms of networking may be enabled not only through the comment section within the published feed during the process of transmitting the feed content, but also through additional interactions. For example, the physician may receive notifications when a patient or another physician “likes” their post, and may respond to such feedback with comments, thereby facilitating social networking across various relationships such as physician-patient, physician-physician, and patient-patient.

Meanwhile, a physician may transmit not only feeds authored by themselves but also feeds authored by other physicians to a target patient. To this end, a bookmark function may be provided to allow the physician to mark and store specific feeds, for example, feeds intended to be sent to their patients at a later time.

The ability to transmit a feed authored by another physician may be selectively enabled or disabled according to MobiDoc™ policy. Since the original author remains identified and the feed is merely forwarded, the forwarding allows the original author to share their ideas, hospital, and identity with patients (MobiDoc™ members). For this reason, MobiDoc™ currently permits such forwarding publication. However, this policy may be modified in the future, for example, by enabling the forwarding only upon the original author's consent, selectively allowing it, or distributing revenue if the forwarding generates income.

### Separate Feed Content That may be Included as Part of CRM Configuration

As illustrated in FIG. 15, a CRM message may include information about a disease, health-related information, or other professional articles sent by a physician 1501 or a hospital to a patient. These articles may be inserted directly, but as illustrated, may also be presented in the form of hyperlinks. The hyperlinks connect to the corresponding MobiDoc™ Feed articles via an app and/or a web. Since MobiDoc™ Feeds may only be authored by licensed physicians, their content may be considered verified. Accordingly, when a physician who treated the patient inserts and sends a professional column article in the form of a feed tailored to the patient's condition and body part as part of CRM, the patient's attention and engagement with the CRM itself may increase. As stated above, conventional CRM messages sent by hospitals via SMS or MMS are often perceived by patients as advertisements and may be disregarded. However, by integrating MobiDoc™ Feeds into a CRM field 1510 as shown in FIG. 15, the patient may perceive that the hospital and physician genuinely care for and manage them as valued customers.

Accordingly, the positive functions of CRM may be effectively realized, enabling the hospital to manage and strengthen its relationship with patient-customers. The enhancement of such CRM functions may be further amplified by integration with MobiDoc™ Clinic, a telemedicine service provided by MobiDoc™.

### Remote Consultation Guidance Included in CRM

In some cases, even when a patient makes a follow-up appointment, the no-show rate may not be low. Various reasons may exist for a patient missing or canceling a scheduled follow-up, including uncertainty about whether the follow-up is truly necessary and/or practical difficulties such as work or childcare obligations. MobiDoc™ Clinic, the telemedicine service provided by MobiDoc™, may function as a nudge within the CRM system, enabling such patients to more easily access follow-up consultations. Even a short remote consultation may allow the patient to re-engage with the physician who performed the initial consultation and discuss the progress of the condition, the current status, or side effects of prescribed medications. As a result, the patient may make an informed decision as to whether an additional in-person hospital visit is required.

Accordingly, according to one embodiment, in a field 1511 of FIG. 15, a guidance indicating that a remote consultation may be requested via MobiDoc™ Clinic and/or a hyperlink for requesting such a remote consultation is provided as part of the CRM.

Meanwhile, the standards for allowing remote consultations may vary depending on the country and the time period. Accordingly, in one embodiment, a CRM to be transmitted at a time when the lawful conditions are met, in accordance with guidance permitted by applicable regulations, may optionally include guidance on a remote consultation and/or a hyperlink for requesting a remote consultation.

For example, the guidance and hyperlink for requesting a remote consultation in the field 1511 may be provided only when the consultation is a follow-up consultation allowed within a predetermined period, for example, within 30 days, as counted backward from the time the illustrated CRM is to be transmitted. For this purpose, D-0 day (the date of the previous reservation) information derived from the hospital's electronic medical record (EMR) and/or prior reservation data may be used and compared with the scheduled time of CRM transmission. In other words, whether the CRM is to be transmitted 7 days or 2 months after the previous consultation may be identified. If remote consultations are permitted only for follow-up consultations within the same hospital and for the same disease within 30 days, then the earlier case (CRM sent 7 days after the prior consultation) may include the remote consultation guidance and link, while the latter (CRM sent after 2 months) may not.

Meanwhile, in the case of D+Day CRMs that provide follow-up care for already conducted consultations or procedures, such guidance and link may also be included only within a predetermined reference period (e.g., 30 days). For example, in creating CRM content for D+1 or D+3 (1 or 3 days after a consultation or procedure date D), the remote consultation guidance and hyperlink may be omitted in cases where the follow-up consultation is no longer permitted (e.g., more than 30 days have elapsed since the last face-to-face consultation).

According to another embodiment, a CRM prompting the patient to request a remote consultation may be transmitted before the expiration of the permissible period for follow-up consultations. For example, such CRM may be sent once on Day 21 and again on Day 28 after the initial consultation or procedure, to guide the patient toward remote follow-up consultation within the permitted timeframe.

### Initiation of MobiDoc™ Clinic (remote Consultation) Via CRM

FIG. 16 illustrates a remote consultation being initiated and carried out via a link in the field 1511.

Although not illustrated, a patient client completes a consultation registration and requests access to a consultation room. The consultation room refers to a session in which the patient waits for a physician to initiate the consultation in a remote consultation process provided through MobiDoc™ Clinic. The consultation room not only indicates that the remote consultation registration has been completed but also functions to ensure that the patient is in a standby state, waiting for the physician to start the consultation.

After the consultation registration is completed, the client associated with the patient account is brought into a consultation room stage upon approval by the hospital and/or physician. This indicates that the service status of the patient client for the corresponding patient account has transitioned from the registration stage to a waiting-for-consultation stage. The consultation room corresponds to a state in which video and/or audio communication between the patient and the physician may be immediately initiated once the physician starts the consultation. This provides a user experience similar to that in an offline hospital, where a nurse guides a patient into a consultation room to wait, and the physician initiates the consultation upon being notified that the patient is ready. In some tertiary hospitals, a physician may alternate between two adjacent (connected) consultation rooms. While the physician is conducting a consultation in one room, a nurse prepares the next patient in the other room. During this time, EMRs, relevant medical images, test results, and other information are preloaded to prevent the physician from wasting time in preparation. The physician completes the consultation and necessary charting in one room, then proceeds to the next room, where the patient and all preparations are ready, to begin the next consultation without delay. MobiDoc™ Clinic provides such a function in a remote consultation context.

Accordingly, by reducing unnecessary time consumption, the physician may substantially focus on the essence of treatment even when consulting the same number of patients, thereby minimizing patient inconvenience caused by consultation delays and enabling the provision of medical services to a greater number of patients. The standby state after entering the “consultation room” of the MobiDoc™ Clinic, as described above, fulfills such a function.

Improvements in consultation efficiency achieved by allowing the patient to remain in a waiting state and ensuring the possibility of immediate commencement of consultation are important, as they provide better service to both medical professionals and patients. In conventional remote medical services, once a patient requests an online (remote) consultation, the physician (or hospital) typically initiates a connection attempt to the patient, such as through a video or voice call. However, since a remote consultation is conducted online and does not involve face-to-face interaction in person, the patient may, while waiting for the consultation, leave the device unattended, engage in other activities, or move away from the device. As a result, when the physician (or hospital) attempts to begin the consultation, the possibility of immediate availability for consultation may not be ensured.

The MobiDoc™ Clinic illustrated in FIG. 16 checks, via the patient client, whether the patient is actually in a ready state for consultation, and thus whether the consultation may be initiated immediately once the physician begins the consultation procedure. It confirms that the patient is in a standby state having entered the consultation room and allows the physician client to recognize this status.

If, as a result of checking the patient client side, it is determined that the patient is on a call, not viewing the screen, away from the device, or performing other tasks on the terminal 101, such that the consultation cannot immediately begin when the physician attempts to start, this situation may be notified to the physician. In addition to merely providing the notification, the system may also remind the physician that “the patient may not be ready for consultation” and may offer the physician an option to consult with another patient first. For the patient, an alert may be provided indicating that “the consultation will begin only when the patient is in a standby state”, thereby producing a nudge effect to ensure the patient is prepared.

With such consideration, the physician may identify, among patients who have completed consultation registration and entered the virtual consultation waiting room, which patients are ready for the start of a consultation, and may begin a consultation by selecting a well-prepared patient. This prevents the physician and hospital from experiencing inefficiencies caused by connection delays or failures. For patients who are not ready, hospital staff or a remote healthcare service provider may guide the patient terminal to prepare for a consultation by notifying the patient that the consultation will only begin when preparation is complete (e.g., keeping the app open, refraining from other tasks, and monitoring the consultation screen). According to the above-described embodiments, the likelihood of initiating a consultation with a patient immediately is increased.

FIG. 16 illustrates an example of a patient terminal 1610 during an ongoing consultation. The patient may be informed that a remote consultation is available for a follow-up visit and thus may receive medical care without physically visiting the hospital. This is particularly effective when the need for a follow-up visit is uncertain, allowing the patient to consult the physician about the course of the condition and adjust medication usage without the burden of an in-person visit. As a result, patients who might have hesitated to pursue a follow-up may be encouraged to continue treatment with the physician, thereby reducing potential gaps in care and preventing deterioration of health.

The examples described herein may be implemented by using a hardware component, a software component, and/or a combination thereof. A processing device may be implemented using one or more general-purpose or special purpose computers, such as, for example, a processor, a controller and an arithmetic logic unit (ALU), a DSP, a microcomputer, an FPGA, a programmable logic unit (PLU), a microprocessor or any other device capable of responding to and executing instructions in a defined manner. The processing device may run an operating system (OS) and one or more software applications that run on the OS. The processing unit also may access, store, manipulate, process, and generate data in response to execution of the software. For purpose of simplicity, the description of a processing unit is used as singular; however, one skilled in the art will appreciate that a processing unit may include multiple processing elements and multiple types of processing elements. For example, the processing unit may include a plurality of processors, or a single processor and a single controller. In addition, different processing configurations are possible, such as parallel processors.

The software may include a computer program, a piece of code, an instruction, or some combinations thereof, to independently or collectively instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of examples, or they may be of the kind well-known and available to those having skill in the computer software arts. Examples of non-transitory computer-readable media include magnetic media such as hard disks, floppy disks, and magnetic tape; optical media such as CD-ROM discs and/or DVDs; magneto-optical media such as optical discs; and hardware devices that are specially configured to store and perform program instructions, such as read-only memory (ROM), random-access memory (RAM), flash memory, and the like. Examples of program instructions include both machine code, such as produced by a compiler, and files containing higher-level code that may be executed by the computer using an interpreter. The above-described devices may be configured to act as one or more software modules in order to perform the operations of the above-described embodiments, or vice versa.

A number of example embodiments have been described above. Nevertheless, it should be understood that various modifications may be made to these example embodiments. Suitable results may be achieved when the described techniques are performed in a different order, and/or if components in a described system, architecture, device, or circuit are combined in a different manner, and/or replaced or supplemented by other components or their equivalents.

Therefore, other implementations, other examples, and equivalents to the claims are also within the scope of the following claims.

1. A computer-implemented method comprising:
providing a first interface configured to generate customer relationship management (CRM) content including text and one or more image in association with a medical institution client assigned to a hospital or a physician;
extracting, by a processor, a tag for an image included in the CRM content while storing the generated CRM content in a storage unit; and
in response to a selection of a recipient and CRM content to be transmitted, transmitting the selected CRM content to the recipient,
wherein extracting comprises obtaining, through a Bidirectional Encoder Representations from Transformers (BERT) model, at least one keyword for the image from the CRM content and extracting the obtained keyword as a tag; and
wherein, the BERT model is trained by assigning a higher weight to a predetermined number of tokens that sequentially follow an image in the CRM content than to remaining tokens.

2. The computer-implemented method of claim 1, further comprising: providing a second interface that is linked with the first interface and configured to provide a function of searching for and selecting an image to be inserted into the CRM content.

3. The computer-implemented method of claim 2, wherein results provided by the image search are filtered based on at least one piece of tag information associated with the image.

4. The computer-implemented method of claim 2, further comprising:
providing a third interface that is distinguished from the second interface and configured to receive a request to create a new image.

5-7. (canceled)

8. The computer-implemented method of claim 1, further comprising:
providing identification information of a patient who has received treatment at the end of treatment and receiving a selection as to whether to transmit the CRM content to the patient.

9. The computer-implemented method of claim 1, wherein the CRM content includes a link for requesting remote treatment, and when the link is selected, a remote treatment request process is initiated.

10. A non-transitory computer-readable storage medium storing instructions that, when executed by a processor, cause the processor to perform the computer-implemented method of claim 1.

11. An electronic device comprising:
a processor;
a communication interface operatively coupled to the processor and configured to provide communication between a medical institution client and a terminal of a recipient; and
a storage unit storing customer relationship management (CRM) content,
wherein the processor is configured to perform the computer-implemented method of claim 1.
